(19) United States
(12) Patent Application Publication
(10) Pub. No.: US 2006/0054720 A1
(43) Pub. Date: Mar. 16, 2006

(54) Ice dam removal system

(76) Inventors: John R. Valiton, Maple Grove, MN (US); Scott B. Rethwill, Blaine, MN (US)

Correspondence Address: SCHROEDER & SIEGFRIED, P.A., 15600 WAYZATA BOULEVARD, SUITE 200, WAYZATA, MN, 55391, US

(21) Appl. No.: 10/937,994

(22) Filed: Sep. 10, 2004

(51) Int. Cl. A61L 9/04; A24F 25/00; A62C 13/62; B05B 7/06; A62C 13/66

(52) U.S. Cl. 239/424.5; 239/302; 239/53; 239/55

(57) ABSTRACT

Apparatus for melting and preventing the formation of roof ice or ice dams on the upper surface of a roof which includes an elongated water-porous container having a closed interior within which a deicing mixture is contained that is composed of a heat-generating deicing agent and a corrosion-inhibiting agent. The container rests on the roof of a building above the ice dam in a transverse, obstructing position relative to the path along which water normally runs so that the water passes through the container and absorbs the deicing mixture to produce a strong deicing brine solution. The brine solution melts any ice it contacts in a medium having a temperature above −15° Fahrenheit, and inhibits corrosion of associated drains, gutters, etc. Heat-absorbing and ecologically friendly deicing agents may also be added to the mixture to prolong the deicing action and aid surrounding vegetation.

### BACKGROUND OF THE INVENTION

Our invention is directed generally toward the field of preventing the formation of roof-ice or ice dams upon shingled rooftops and the consequential damage associated therewith. More particularly, our present invention is directed toward an improved ice melting apparatus that utilizes a generated brine solution with environmentally friendly corrosion inhibiting elements to melt and/or prevent the build-up of ice upon the roof in the event of an occurring ice dam. In this respect, the present invention constitutes a substantial improvement over methods and apparatus previously known and used, in that it operates more effectively and eliminates much of the damage caused thereby.

Ice dams tend to build up on the overhang or eaves of a building adjacent the areas of roof drains and gutters. This is so because the eaves are generally less insulated and situated at the lower edge of the roofline where less heat is stored. Consequently, as heat rises within the building, snow and/or ice present on the upper portion of the roof tends to melt and run down the roof to the eaves where it re-freezes, thus causing the formation of an ice build-up. As this process continues, ice dams form and the water running down the roof begins to pool and back up underneath the roof shingles, where it then enters the building causing significant damage to the inner wall structures. As ice continues to collect and block the drains, more water collects and more ice forms, causing the damage to multiply.

Currently there are only a few known options available to remove ice dams, most of which have either met with little or no success, or are too costly to implement to be practical for the average homeowner. For instance, manually hammering or chipping the ice dams away has been previously contemplated. Also, removal methods utilizing electric cables, steam blasting, and torching have also been employed in the past. Not only are such options time consuming and expensive to implement, none are recommended by roofing authorities. As noted by the Candia Asphalt Shingles Roofing Manufactures Association, all such methods may result in significant permanent damage to the roof shingles and/or involve numerous safety hazards (electrical shock, fire, etc.).

A more recent and related attempt to solve the ice dam problem is set forth in U.S. Pat. No. 6,282,846 issued May 26, 1999 to Nocella, and entitled Roof Drain De-Icer Apparatus. In this apparatus, salt is utilized as a means of deicing with some degree of success adjacent the source of difficulty. It ignores, however, the fact that salt brine is very corrosive to metal gutters, drain pipes, and other metal structures with which it comes in contact. It also fails to take account of the significant damage that salt (i.e., sodium chloride) can cause to nearby grass, trees, shrubbery and all other plant life. Unless additional and expensive care is taken of the salt brine which is generated by the Nocella invention, substantial damage will result to the lawns, trees and shrubbery adjacent its use, since almost all drains discharge at their lesser end, and the salty water reaches the areas where such vegetation is normally located.

In addition to the above, the Nocella device is limited in its applicability and effectiveness in that the brine solution generated from common rock salt will not melt ice in temperatures lower than about 15-20° Fahrenheit (F.). Also, since salt it is a solid in its natural state, it must absorb heat from the environment upon transforming to a brine solution, thereby making it a relatively slow acting agent. As such, it is less reliable in cases of emergency.

It is therefore evident that there is a significant need for a means of effectively removing and preventing the formation of ice dams which is capable of doing so rapidly without causing damage to the shingles and/or creating safety hazards, and which is less corrosive and more friendly to the environment. It is with this object in mind that we have conceived of our present invention, which obviates the need and damaging effects associated with the sole use of salt as described above.

### BRIEF SUMMARY OF THE INVENTION

As described herein, our invention contemplates the use of an aggressive, less corrosive chemical mixture to melt ice dams. This mixture, which is intended to be encased within a porous woven fabric container (described hereafter), is comprised of one or more aggressive yet less corrosive heat-generating deicing agents, such as calcium chloride or magnesium chloride, in combination with a corrosion-inhibiting agent, such as zinc sulfate, to generate a fast acting ice-melting compound that deters corrosion of metal gutters and drain pipes during use. To this mixture may be added a slower acting and more environmentally friendly deicing agent, such as potassium chloride, and the more common yet slower acting sodium chloride, the negative effects of which are largely offset by the added corrosion inhibitor and more environmentally friendly deicing agent.

The more aggressive calcium chloride and magnesium chloride deicing agents rapidly absorb moisture and emit heat upon transforming to a brine solution, and will effectively melt ice at a substantially lower temperature than sodium chloride. With the added slower acting agents, potassium chloride and sodium chloride, the resulting mixture will first aggressively attack the ice dam to create a strong brine solution that will further melt the ice, create more water, and form more deicing brine. The water generated will also contact and absorb the slower acting deicing agents to create a more sustained, long lasting deicing effect that will be effective down to at least −15° F.

Although each of the above chloride deicing agents can be corrosive to some extent with prolonged exposure to metal surfaces, calcium chloride and magnesium chloride are significantly less corrosive than sodium chloride, and have the added benefit of being more aggressive deicing agents. Therefore, even when mixed with sodium chloride, the resulting mixture will also be less corrosive. By further supplementing the mixture with a corrosion inhibitor, such as zinc sulfate, the corrosive effects of the above mixture may be substantially offset. While it is certainly contemplated that the corrosion inhibitor may be added in any form, to provide the most uniform application, it is preferred that the resulting mixture of deicing agents be sprayed with a liquid zinc sulfate, thereby effectively coating or encapsulating each granular component of the deicing mixture with a corrosion-inhibiting layer.

With the exception of common rock salt, each of the above deicing chemicals can be found to some extent in agricultural applications, and if not applied in abundance, should not damage nearby plants and shrubs. Potassium chloride, in particular, is commonly found in many fertilizer applications, and provides the specific purpose in our invention of counteracting the ill effects that sodium chloride may have on surrounding vegetation.

In the preferred embodiment, it is contemplated that the above mixture be sealed within a flexible elongated water-porous bag constructed of a woven geosynthetic fabric. This bag may then be placed on or secured to the rooftop immediately above the ice dam in a transverse, water-obviating position, across the paths of water that normally descend along the upper surface of the roof As a consequence, a substantial portion of such water passes through the water-porous container of the chloride-zinc sulfate mixture and gradually dissolves a portion thereof This, in effect, creates a strong brine solution which depresses the freezing point of the ice dam with which it makes contact, causing same to melt.

With the added corrosion inhibitor, the potentially damaging effect of the deicing brine solution on gutters, drain pipes, etc. is significantly reduced. Moreover, by adding potassium chloride to the mixture, the potentially ill effects caused by any sodium chloride are offset, thereby rendering the brine solution relatively harmless if and when it contacts grass, trees, or shrubbery.

While it is preferred that all deicing agents be utilized in granular form, it is further contemplated as an alternative that the magnesium chloride may be sprayed on the mixture in liquid form, similar to the zinc sulfate. In such case, it is contemplated that the magnesium chloride and zinc sulfate be mixed together and thereafter sprayed on the remaining granules and allowed to dry, effectively coating each of the granules therewith.

Because the container holding the deicing mixture is constructed of a porous woven material, residual minute particles and/or dust from the granular mixture may have a tendency to escape the container during transportation and storage. In order to prevent the escape of such particles and create less mess, a non-porous outer plastic or poly bag is provided within which the porous container may be stored prior to use. This outer bag may be sealed at both ends to completely encapsulate the deicing apparatus, and prevent undesirable leakage of chemicals therefrom.

### BRIEF DESCRIPTION OF THE DRAWINGS

These and other objects and advantages of the invention will more fully appear from the following description, made in connection with the accompanying drawings, wherein like reference characters refer to the same or similar parts throughout the several views, and in which:

FIG. 1 is a perspective view of our improved ice dam removal system incorporating a unique deicing mixture encased in a porous woven fabric container in accordance with our invention;

FIG. 2 is a side elevational view showing the manner in which the porous container of our improved ice dam removal system may be loaded with the deicing mixture;

FIG. 3 is a exploded perspective view showing the manner in which the porous fabric container carrying the deicing mixture of our invention may be inserted in an outer solid polymeric bag for storage and transportation; and

FIG. 4 a partial side elevational view of the roof portion of a building, showing the manner in which our improved ice dam removal system may be used in a typical deicing application.

### DETAILED DESCRIPTION OF THE INVENTION

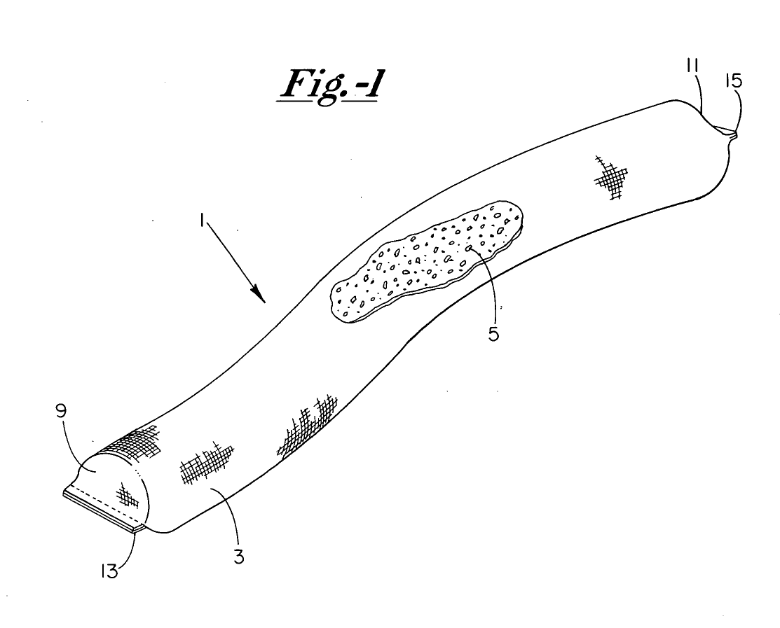

As shown in FIG. 1, our improved ice dam removal system 1 is comprised generally of an outer porous container 3 that is preferably constructed as an elongated tubular structure having a closed interior that encases our improved deicing mixture 5, which will be described in more detail hereafter. As seen, the porous container 3 is constructed of a flexible cotton or polymeric woven fabric which is designed such that the interstices defined by the interwoven strands or yarn are sufficiently small to retain the granular deicing mixture 5 therein, yet sufficiently sized to accommodate and allow water to flow freely therethrough.

In the preferred embodiment, the fabric utilized for container 3 is composed of high-tenacity monofilament polypropylene yarns, which are substantially inert to biological degradation and resist naturally encountered chemicals, alkalines, and acids. Such polypropylene yarns are woven into a stable network such that the yarns retain their relative position an structural integrity. One such material found suitable for meeting these general requirements is the Mirafi® Filterweave® fabric manufactured by TC Mirafi Engineering Services, Inc., 365 South Holland Drive, Pendergrass, Ga. 30567. The porosity of the woven fabric utilized in the manufacture of container 3 should be sufficient to provide a liquid flow rate therethrough which is greater than approximately 70 gal/min/ft², and preferably in the range of 100-150 gal/min/ft². It has been found that the Mirafi® Filterweave® 402 fabric will suitably accommodate the above desired characteristics.

To form container 3 into the desired tubular configuration shown in FIG. 1, it is contemplated that a generally rectangular sheet of the above material be rolled in such manner that the opposite elongated sides of the fabric may be stitched or sewn together to form an internal or external seam (not shown) extending the length thereof. In order to complete the construction of container 3 with a closed interior for carrying the deicing mixture 5, each of the opposite ends 9 and 11 of container 3 are also stitched or sewn along lines 13 and 15, respectfully. Depending on the application for which our ice dam removal system is to be used, it is contemplated that the porous container 3 be constructed in the range of twelve (12) to eighty (80) inches in length, and anywhere from two (2) inches to six (6) inches in diameter.

In the preferred embodiment, the deicing mixture 5 is comprised of one or more relatively aggressive yet less corrosive heat-generating deicing agents, such as calcium chloride or magnesium chloride, in combination with at least one corrosion-inhibiting agent, such as zinc sulfate. In the preferred embodiment, it is contemplated that the deicing agents be of a solid granular or pellet form and the corrosion inhibiting agent, zinc sulfate, be applied as a liquid spray so as to substantially coat or encapsulate each of the deicing granules. It will be appreciated, however, that the various components of the deicing mixture may also be supplied in different forms, without departing from the invention. For instance, it is contemplated that magnesium chloride, if used, may be combined with the zinc sulfate as a liquid and applied to the remaining granular deicing agents in the form of a liquid spray that will dry and adhere thereto.

Since calcium chloride and magnesium chloride are liquids in their natural state, such chemicals have an affinity to return to a liquid. As such, when granular forms of these agents are used, upon contacting ice, snow or water, they rapidly absorb moisture and emit heat in the process of forming a strong deicing brine solution that will aggressively attack and melt the ice dam. Calcium chloride and magnesium chloride are also more aggressive deicing agents in that they will effectively melt ice in temperatures well below that of common rock salt (−25° F. and 5° F., respectively).

To this mixture may be added one or more slower acting deicing agents, such as potassium chloride and/or the more common sodium chloride. These deicing agents will also transform and become a part of the brine solution upon contact with moisture, but in so doing, must absorb heat from the surrounding environment, and are therefore much slower reacting. Notably, in contrast to prior art devices, the negative environmental effects commonly associated with the use of sodium chloride are largely off-set by other elements contained in the above deicing mixture. For instance, with the exception of sodium chloride, each of the above deicing agents are commonly used to some extent in agricultural applications. Potassium chloride, in particular, is commonly used as a fertilizer for vegetation, and will therefore help to counter the potential for damage to the surrounding vegetation, such as grass, trees, and shrubs which may come in contact with the discharged brine solution. Also, the specific inclusion of zinc sulfate as a corrosion inhibiting agent will help counter the corrosive effects of the remaining deicing agents, particularly that of the sodium chloride.

By utilizing the above mixture of aggressive heat-generating deicing agents, in combination with the slower acting heat-absorbing deicing agents and corrosion inhibitor, an improved deicing mixture 5 is formed that will effectively and aggressively melt ice in temperatures down to at least −15° F. Upon absorbing moisture, the resulting deicing mixture 5 will first aggressively attack the ice with which it makes contact, creating a strong brine solution composed of the above chemicals. Such solution will thereafter continue to melt the ice, thereby creating more water, and forming additional deicing brine. While the more aggressive heat-generating deicing agents react quickly to absorb moisture and create more water, the slower acting deicing agents will also be absorbed by passing water and create a more sustained, long-lasting deicing effect.

While it is certainly contemplated that the various deicing agents may be mixed in any of a number of different proportions, it has been found that the following proportion of the constituent parts works well to accomplish the desired deicing function and adequately protect against any negative corrosive and environmental effects: calcium chloride (10%); magnesium chloride (10%); potassium chloride (10%); and sodium chloride (70%). With the above mixture of deicing agents, liquid zinc sulfate may be applied to the mixture in the form of a spray so as to substantially coat or encapsulate the granular mixture therewith. In the preferred formulation, it is contemplated that the corrosion inhibitor will make up approximately five per cent (5%) by total weight of the entire mixture.

While in the preferred embodiment it is contemplated that the magnesium chloride will be added in granular form, it is conceivable that a liquid magnesium chloride may be utilized and applied to the remaining granular mixture by spray application, similar to the zinc sulfate. In such case, it is preferred that the liquid magnesium chloride and zinc sulfate be mixed together to form a liquid compound and sprayed over the remainder of the mixture simultaneously.

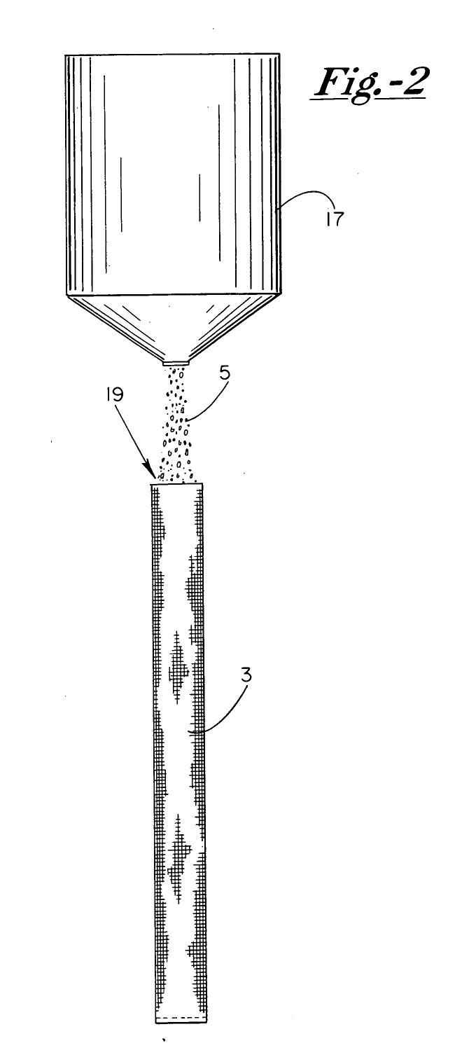

As shown best in FIG. 2, once any liquid components are sprayed on and allowed to dry, the resulting mixture may be loaded into a hopper 17, which may be utilized to automatically feed and load the desired amount of deicing mixture 5 into a plurality of porous containers 3 on a production scale basis. As seen in FIG. 2, notably, each porous container 3 is initially provided with a opening 19 at one end thereof which allows the granular deicing mixture 5 to be loaded into the interior thereof. Once each container 3 is loaded with the desired amount of deicing mixture 5, the container is sealed shut through a secondary sewing or stitching operation to close the open end 19 thereof, as shown in FIG. 1.

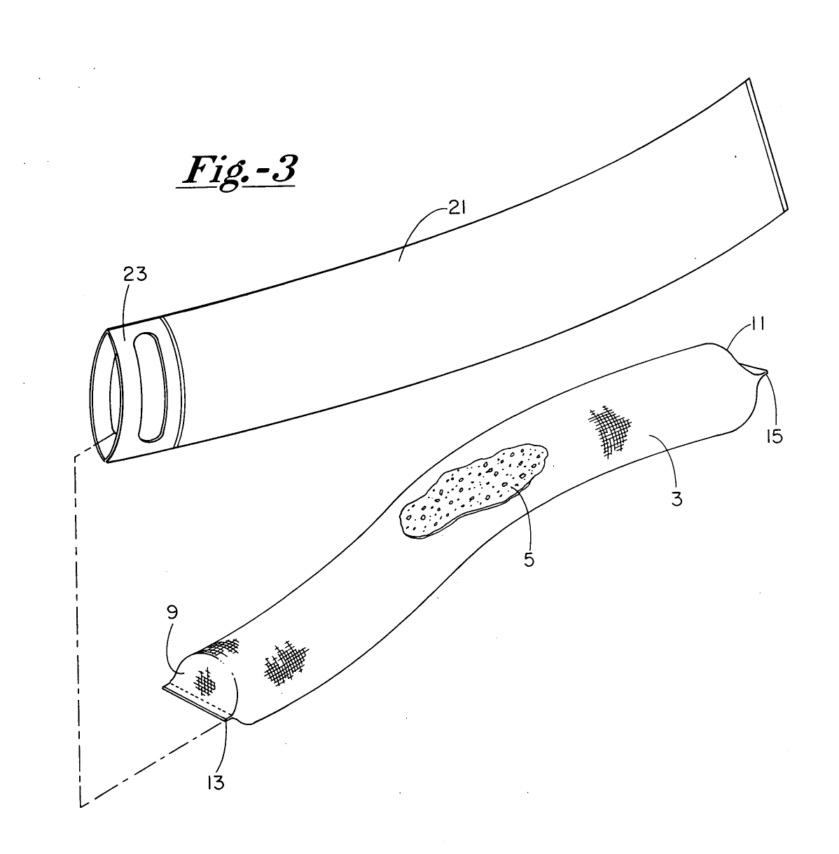

With reference to FIG. 3, it is seen that each porous container 3, upon being filled with deicing mixture 5, may optionally thereafter be loaded into an outer non-porous bag 21 which is preferably constructed of a polymeric material having suitable strength to retain the weight of a loaded container 3. In practice, it has been found that a solid polymeric bag of approximately 4.5 mil thickness is suitable for the above-described purpose. As seen, such a bag 21 preferably conforms to the overall shape and size of the porous container 3 to be stored therein. The polymeric bag 21, as shown, is ultimately sealed at both ends to hold the container 3 therewithin, and a handle 23 is provided on one end for ease of transporting the finished product. By utilizing a solid polymeric bag 21 for storage and transportation of the porous container 3, residual minute particles and/or dust generated from the granular mixture therein will remain confined within the sealed container 21, thereby preventing the undesirable leakage and mess created from the escape of such chemicals.

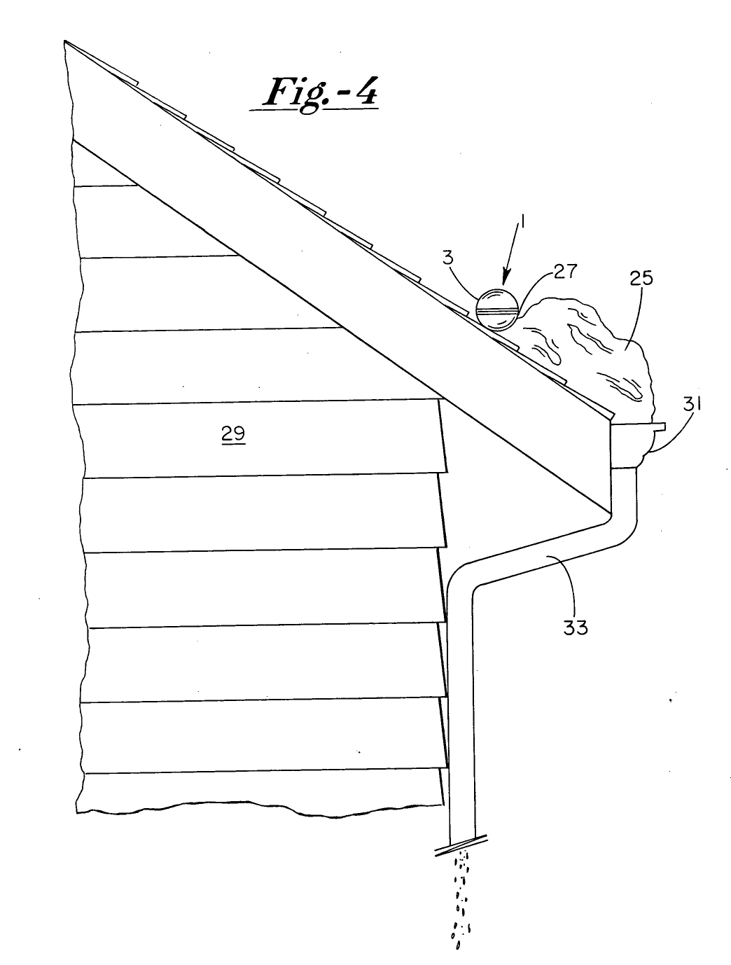

In use, as shown in FIG. 4, our improved ice dam removal system may be placed on any rooftop experiencing the formation of an ice dam 25 at a point immediately adjacent the upper edge 27 thereof. Although it is possible to externally secure container 3 in a position adjacent the ice dam 25, it is deemed preferable to allow the ice dam removal system 1 to physically abut and rest against the ice dam, as shown in FIG. 4. In such case, the ice dam will effectively retain the container 3 in proper position adjacent the upper edge 27 thereof As shown, the flexible woven material of container 3 and deicing mixture 5 therewithin will conform generally to the shape of the upper edge 27 of ice dam 25, as it rests thereagainst.

As heat rises within the building structure 29, the upper portions of the roof are warmed, consequently causing snow and/or ice thereon to melt and run downwardly toward the ice dam removal system. Due to the porous nature of the container 3, water running down the roof will enter the container and begin absorbing the granular deicing mixture 5 contained therewithin. Upon absorption of the deicing agents, a strong brine solution is created which comes in contact with the ice dam 25, thereby effectively depressing the freezing temperature of such ice and causing same to melt. Such brine solution will effectively create channels through the ice dam, causing further melting thereof and drainage into the gutter 31. Because the brine solution also contains an effective corrosive inhibitor, the potential damaging effects of the sodium chloride and/or other agents are minimized. As the solution and melted ice flow through and out the discharge end of drain pipe 33, any ill effects of the vegetation normally caused by the presence of sodium chloride are also minimized due to the added fertilization effects caused by the inclusion of potassium chloride.

With our improved ice dam removal system 1, it is seen that the formation of ice dams upon roofs may be effectively removed and prevented without causing damage to the shingles or creating safety hazards, as commonly associated with conventional manual techniques for removal of such ice dams. Our new ice dam removal system 1 provides a unique and inexpensive means for aggressively attacking and preventing ice formations upon roofs, and utilizes an aggressive yet less corrosive deicing chemical mixture that inhibits corrosion of metal gutters and drains, and counters the negative environmental impact commonly associated with other conventional chemical deicing apparatus.

It will, of course, be understood that various changes may be made in the form, details, arrangement and proportions of the parts without departing from the scope of the invention which comprises the matter shown and described herein and set forth in the appended claims.

1. A deicing apparatus for melting and preventing the formation of ice on the upper surface of a roof, comprising:
(a) an elongated water-porous container having a closed interior; and
(b) a deicing mixture contained within said closed interior of said container, said mixture being comprised of a heat-generating deicing agent and a corrosion-inhibiting agent;
(c) said container being constructed to allow passage of water therethrough to absorb said mixture and produce a deicing solution that will depress the freezing point of the ice formations on the upper surface of the roof so as to melt same.

2. The deicing apparatus defined in claim 1, wherein said heat generating deicing agent is comprised of calcium chloride.

3. The deicing apparatus defined in claim 1, wherein said heat generating deicing agent is comprised of magnesium chloride.

4. The deicing apparatus defined in claim 1, wherein said corrosion-inhibiting agent is comprised of zinc sulfate.

5. The deicing apparatus defined in claim 1, wherein said container is constructed of a material that is substantially inert to biological degradation.

6. The deicing apparatus defined in claim 5, wherein said container is constructed of a plastic woven fabric.

7. The deicing apparatus defined in claim 1, wherein said container is constructed of a fabric composed of woven polypropylene yarns.

8. The deicing apparatus defined in claim 1, wherein said container is constructed of a woven fabric.

9. The deicing apparatus defined in claim 1, wherein said container is constructed of a woven fabric, the porosity of which provides a flow rate therethrough at least as great as 100 gal/min/ft².

10. The deicing apparatus defined in claim 1, wherein said container is constructed of a polypropylene woven fabric that is substantially inert to biological degradation, and the porosity of which provides a flow rate therethrough at least as great as 100 gal/min/ft².

11. The deicing apparatus defined in claim 1, wherein said mixture includes a heat-absorbing deicing agent.

12. The deicing apparatus defined in claim 11, wherein said heat-absorbing deicing agent is comprised of potassium chloride.

13. The deicing apparatus defined in claim 11, wherein said heat-absorbing deicing agent is comprised of sodium chloride.

14. A deicing apparatus for melting and preventing the formation of ice on the upper surface of a roof, comprising:
(a) an elongated water-porous container having a closed interior, said container being constructed of a plastic woven fabric; and
(b) a deicing mixture contained within said closed interior of said container, said mixture being comprised of plurality of heat-generating deicing agents, a plurality of heat-absorbing deicing agents, and a corrosion-inhibiting agent;
(c) said container being constructed to allow passage of water therethrough to absorb said mixture and produce a deicing solution that will depress the freezing point of the ice formations on the upper surface of the roof so as to melt same.

15. The deicing apparatus defined in claim 14, wherein said plurality of heat-generating deicing agents include calcium chloride and magnesium chloride, said heat-absorbing deicing agents include potassium chloride and sodium chloride, and said corrosion-inhibiting agent includes zinc sulfate.

16. The deicing apparatus defined in claim 14, wherein said mixture is composed of about 20% heat-generating deicing agents.

17. The deicing apparatus defined in claim 14, wherein said mixture is composed of about 80% heat-absorbing deicing agents.

18. The deicing apparatus defined in claim 14, wherein said container is constructed of polypropylene yarn woven in such manner as to provide a water flow rate therethrough that is greater than about 100 gal/min/ft².

19. The deicing apparatus defined in claim 14, wherein said corrosion-inhibiting agent is applied to said mixture in liquid form.

20. The deicing apparatus defined in claim 19, wherein said corrosion-inhibiting agent is sprayed on said mixture.

21. The deicing apparatus defined in claim 14, wherein said deicing agents of said mixture are granular in form and said corrosion-inhibiting agent is a liquid sprayed-on coating applied to said granular deicing agents.

22. The deicing apparatus defined in claim 14, wherein said deicing mixture has an effective deicing operating range extending as low as approximately −15° F.

23. The deicing apparatus defined in claim 14, including an outer non-porous plastic container within which said porous container may be stored during non-use.

24. The deicing apparatus defined in claim 23, wherein said outer non-porous container constitutes a sealable polymer bag having a carrying handle at one end thereof

25 A method for melting and preventing the formation of ice on the upper surface of a roof, comprising the steps of.
(a) providing a water-porous container having an interior with a closable opening thereto;
(b) loading a deicing mixture comprised of a heat-generating deicing agent and a corrosion-inhibiting agent through said opening and into said interior of said container;
(c) closing said opening of said container;
(d) placing said container on a roof above an ice formation so as to allow water running down said roof to pass through said porous container and absorb said mixture and produce a deicing solution that will depress the freezing point of said ice formation and melt same.
